(19) United States
(12) Patent Application Publication
(10) Pub. No.: US 2024/0418810 A1
(43) Pub. Date: Dec. 19, 2024

(54) LOCAL COIL WITH ENERGY-SAVING APPARATUS

(71) Applicant: Siemens Healthineers AG, Forchheim (DE)

(72) Inventor: Jan Bollenbeck, Eggolsheim (DE)

(21) Appl. No.: 18/740,098

(22) Filed: Jun. 11, 2024

(30) Foreign Application Priority Data
Jun. 16, 2023 (DE) 10 2023 205 673.9

(51) Int. Cl. G01R 33/36

(52) U.S. Cl. CPC G01R 33/3692

(57) ABSTRACT

a local coil for a magnetic resonance tomography system and a magnetic resonance tomography system with a local coil. The local coil has a detuning apparatus with a signal path for activation. The local coil has a further functional unit with two operational states having differing energy usage. The local coil further has an energy usage control system which is configured to control the operational states of the functional unit dependent upon an image acquisition.

### CROSS REFERENCE TO RELATED APPLICATIONS

This application claims the benefit DE 10 2023 205 673.9 filed on Jun. 16, 2023, which is hereby incorporated by reference in its entirety.

### FIELD

Embodiments relate to a local coil for a magnetic resonance tomography system and a magnetic resonance tomography system with a local coil. The local coil has a detuning apparatus with a signal path for activation.

### BACKGROUND

Magnetic resonance tomography systems are imaging apparatuses that, to map an examination object, align nuclear spins of the examination object with a strong external magnetic field and by way of an alternating magnetic field, excite them into precession about this alignment. The precession and/or the return of the spin from this excited state into a state with lower energy itself generates an alternating magnetic field as the response, that is received via antennas.

With the aid of magnetic gradient fields, a position encoding is impressed upon the signals, that subsequently provides an allocation of the received signal to a volume element. The received signal is then evaluated and a three-dimensional imaging representation of the examination object is provided. For receiving the signal local receiving antennas, referred to as local coils, are used that, in order to achieve a better signal-to-noise ratio, are arranged directly on the examination object.

The local coils are usually connected via coaxial cables to receivers of the magnetic resonance tomography system. However, the management of the cables is difficult due to the thickness and the required sheath wave filter and the multipole plug connection is fault-prone.

Another approach is to transfer the magnetic resonance signals wirelessly from the local coil to the receiver. However, a consumers such as analog amplifiers have a significant energy usage due to the required dynamic precondition for this is that the energy supply for the local coil is also provided wirelessly and/or via energy stores such as batteries in the local coil. Therein, for example, with limited essential range.

Regardless of the energy usage, with a line-bound local coil, the waste heat generated thereby on the patient is also a problem.

### BRIEF DESCRIPTION AND SUMMARY

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments improve the operation of a local coil.

A local coil is provided for use with a magnetic resonance tomography system. Embodiments may be used in combination with a wireless local coil. A local coil that includes no connection by a line to the magnetic resonance tomography system, whether an electrical connecting line or an optical connecting line, is designated a wireless local coil. Using a wireless local coil, the management of the local coil is significantly simplified. A wireless local coil necessarily includes a wireless energy supply, such as a battery or a supercapacitor, in order to enable interruption-free operation. A wireless local coil benefits from a lower energy usage with a longer uninterrupted useful life, as described below. But local coils with a line-bound connection to the magnetic resonance tomography system also benefit, for example, from reduced heating.

The local coil includes a detuning apparatus with a signal path for activation. An apparatus that is configured to protect an antenna coil for receiving a magnetic resonance signal of the local coil in an active state against damage by an excitation pulse for the nuclear spin is designated a detuning apparatus. In order to generate the magnetic resonance signal in the body of a patient, initially an excitation of the nuclear spin is required. This takes place by way of a radio frequency signal that, due to the constant static magnetic field B0, also has substantial signal portions with the Larmor frequency that also have the magnetic resonance signals that are to be received by the antenna coil. The excitation pulse may have power levels of several hundred watts or a few kilowatts, so that with a resonantly tuned antenna coil, the induced currents and voltages might damage the antenna coil and endanger the patient. In order to prevent this, the detuning apparatus is activated before the emission of the excitation pulse via the signal path, so that the antenna coil is no longer resonant at the Larmor frequency and the induced currents and voltages are orders of magnitude smaller.

In a local coil, the signal path for activating the detuning apparatus is either line-bound or, for example, in a wireless embodiment, wireless.

In the line-bound local coil, a separate line for transferring a detuning signal may be provided for activating the detuning apparatus. For example, a signal line that transfers a current and/or a voltage that activates and/or directly operates the detuning apparatus is conceivable. A line that transfers an activation command may be used.

In the wireless embodiment of the local coil, it may be provided, for example, that as the detuning signal, a radio frequency magnetic or electromagnetic alternating field from the magnetic resonance tomography system is induced at a frequency not equal to the Larmor frequency, that is received by an antenna or antenna coil of the local coil and activates the detuning apparatus. The detuning signal may itself transfer enough energy to supply the detuning apparatus with energy. For example, the detuning signal received by the antenna may be rectified and may thus supply and activate the detuning apparatus. The detuning signal may serve only as a message on the basis of which the detuning apparatus actively carries out the activation itself while using an energy source of the local coil, such as for example, a battery. Then the signal may, for example, also be transferred optically.

The local coil includes at least one further functional unit that has two operational states with differing energy usage. This may be, for example, low noise amplifiers (LNAs) or mixers for downmixing the received magnetic resonance signals. In the simplest case, the operational states may be a switched-on state when magnetic resonance signals are to be processed and a switched-off state in pauses therebetween. Magnetic resonance signals for a mapping only occur in the context of an image acquisition sequence, for example time periods, for example, when the nuclear spins in a volume to be acquired are in phase. In these re-phased portions, signals with particularly large amplitudes may occur so that LNAs and/or mixers for linear processing with high dynamic responses must be operated with particularly large currents, whereas in portions with small amplitude, smaller currents are also sufficient, that is a further operational state.

A digital local coil, for example, digital units such as multiplexers, A/D converters, memory stores and/or processors may be activated with evaluable magnetic resonance signals only in the corresponding phases, whereas therebetween they remain in an energy-saving rest mode.

The local coil includes an energy usage control system. This is to be understood as a circuit, analog or digital, or a processor that is configured to control the operational states of the functional unit and/or of the functional units dependent upon an image acquisition. In other words, the energy usage control system sets the functional unit, dependent upon the image acquisition, for example upon a progression of an image acquisition sequence, in different operational states with different energy usage. The energy usage control system sets the functional unit into the operational state with the lowest energy usage that is possible in the respective portion of the image acquisition sequence.

In an embodiment, by way of the energy usage control system, the local coil attains a reduced energy usage and thus an extended operating time with the energy that is available.

In an embodiment of the local coil, the energy usage control system is configured to acquire a detuning signal for the detuning apparatus on the signal path. In the simplest case, the energy usage control system may be in direct signal connection with the signal path, for example, it may have a signal input that also receives the detuning signal for the detuning apparatus fed to it via an electrical connection. Also possible, however, is a processing of the detuning signal, such as voltage conversion, filtering, or level limitation.

The energy usage control system then controls the operational states of the functional unit dependent upon the detuning signal. In the simplest case, the energy usage control system may, for example, interrupt the energy supply to the amplifier and/or the mixer if the detuning signal specifies an activation of the detuning apparatus, since during the excitation pulse, no receiving of a magnetic resonance signal is possible and they may be deactivated. The energy usage control system may acquire and take account of further signals with information regarding the image acquisition, such as, for example, described below, signals of a sensor to acquire the gradient fields. It is also possible to take into account additional information regarding the temporal progression by way of the energy usage control system so that the change in the operational states are caused by the detuning signal, but do not necessarily take place synchronously therewith.

By way of the coupling of the detuning signal and the operational state, an energy saving may be realized in a simple manner. Furthermore, however, the energy usage control system may also further reduce the energy usage, given knowledge of the image acquisition sequence, for example, via portions without a magnetic resonance signal, in that even beyond the immediate duration of the detuning signal, but triggered by it, an operational state with a reduced energy usage is set. By way of the detuning signal for a pre-determined time, a pre-determined operational state may be set by the energy usage control system, for example, to receive the magnetic resonance signals generated by the excitation pulse associated therewith.

In an embodiment of the local coil, the energy usage control system includes a decoder. The decoder is configured to decode a large number of messages encoded in the detuning signal. A large number is therein regarded as a quantity of messages that goes beyond binary input/output information of the detuning signal for controlling the detuning apparatus. For example, by way of one or more short interruptions of the detuning signal, further information is encoded into the detuning signal. Therein, the interruptions are so short that, for example, by way of a low pass filter in the detuning apparatus they do not lead to a change in the detuned state or are already eliminated in the decoder by way of a holding element, before the detuning signal is passed on by the decoder to the detuning apparatus.

The detuning signal may also be understood herein in the broad sense as a signal that is transferred on the same line and/or signal path as the actual signal triggering the detuned state of the detuning apparatus. For example, by way of a detuning signal and/or a signal on the signal path for the actual detuning signal may transfer a message at a time point without an excitation pulse. Preferably, this encoded message differs from the actual detuning signal in that it does not trigger and/or activate the detuning apparatus, for example, in that it includes short pulses, as compared with the actual detuning duration, that may also be emitted by the magnetic resonance tomography system before the excitation pulse.

The energy saving apparatus receives the message and sets a plurality of operational states of the functional unit dependent upon the messages. For example, additional operational states with a different energy saving level are possible. This may be, inter alia, a message relating to an expected amplitude of the magnetic resonance signal and/or of a bias current in the LNA in order to be able still to process the signal in the linear region. A message relating to a duration of the energy-saving state that deviates from the duration of the detuning signal may be used.

In the magnetic resonance tomography system with a corresponding local coil, the magnetic resonance tomography system includes a corresponding and/or complementary encoding unit in order to encode the message regarding the operational state into the detuning signal and subsequently to transfer it to the local coil.

The magnetic resonance tomography system with the encoding unit may transfer further energy saving measures via the detuning signal to the local coil by an encoded message and a decoder in the local coil and so further improve the run time. The use of the detuning signal therein saves additional communication channels and/or lines and simplifies, for example with a wireless local coil, both the authorization of the system as well as the structure of the local coil, that implies further energy savings.

In an embodiment of the local coil, the energy usage control system is configured, in the absence of a predetermined image acquisition event, to set the functional unit and/or functional units in a predetermined operational state. The absence of a predetermined image acquisition event may be considered to be, for example, the lack of the detuning signal from gradient fields, as described below in relation to the magnetic field sensor, or other signals that may be acquired by the local coil in a predetermined interval. If, for example, no excitation pulse has been emitted for a relatively long time, i.e., in a period of a plurality of seconds to minutes and if the local coil has acquired no detuning signal correlated therewith, then no excited nuclear spin is present in the sample or in the patient and no further magnetic resonance signal may be received. For example, the LNA and/or mixer of the energy usage control system may be set into a rest state and/or switched current-free. By way of a renewed detuning signal, the energy saving state of the energy usage control system may be ended again. The energy-saving operational state may remain for a predetermined time.

The recognition of operational pauses through the absence of signals provides further energy-saving time periods for the local coil.

In an embodiment of the local coil, the local coil includes a magnetic field sensor in signal-conducting connection with the energy usage control system. The magnetic field sensor is configured to acquire a gradient field. Examples of magnetic field sensors are induction coils or Hall sensors.

In principle, the induction coils acquire only varying magnetic fields, whereas the static B0 field is already blocked out. If the signal from the induction coil is passed through a low pass filter, then only signals induced by the gradient fields remain, so that they may be easily detected. Hall sensors may become saturated by the B0 field, that makes the detection of the gradient fields difficult. This problem may be solved by way of suitable alignment and/or configuration, so that the B0 detection may then also be used, for example, by way of the energy usage control system to set the local coil outside the patient tunnel into an energy-saving operational state.

The energy usage control system is therein configured to set the operational states dependent upon a signal from the magnetic field sensor. It is thus conceivable, for example, that in the absence of the detuning signal during particular time portions of an image acquisition sequence that may be defined by a temporal relation to the switching processes of the gradient fields, also no magnetic resonance signals are present and an energy saving operational state is activated for the functional unit by the energy usage control system. It would also be conceivable to create an operational state with an increased energy usage and associated receiving readiness if gradient fields are active, since they are required in the associated sequence for readout.

### BRIEF DESCRIPTION OF THE FIGURES

The above-described properties, features and advantages and the manner in which they are achieved are made more clearly and distinctly intelligible in conjunction with the following description of the embodiments that are set out in greater detail making reference to the drawings.

FIG. 1 depicts a schematic representation of a magnetic resonance tomography system according to an embodiment.

FIG. 2 depicts a schematic representation of a temporal progression of an image acquisition sequence according to an embodiment.

FIG. 3 depicts a schematic detailed representation of an embodiment of a local coil.

FIG. 4 depicts a schematic representation of a temporal progression of an image acquisition sequence according to an embodiment.

FIG. 5 depicts a schematic detailed representation of an embodiment of a local coil.

### DETAILED DESCRIPTION

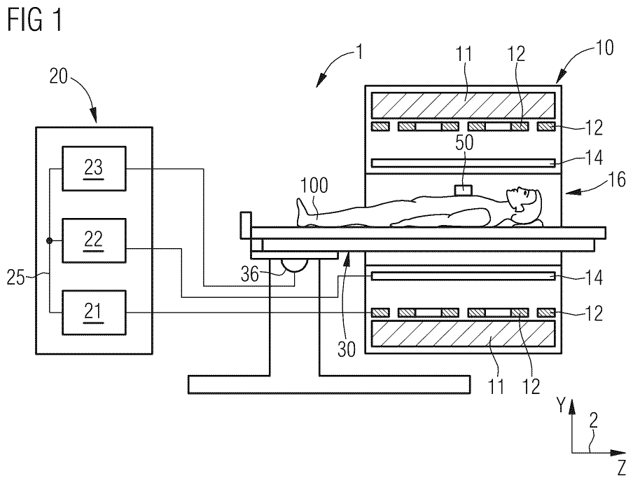

FIG. 1 depicts a schematic representation of an embodiment of a magnetic resonance tomography system 1.

The magnet unit 10 includes a field magnet 11 that generates a static magnetic field B0 for aligning the nuclear spins of samples and/or patients 100 in a scanning region. The scanning region is arranged in a patient tunnel 16 that extends in a longitudinal direction 2 through the magnet unit 10. A patient 100 is movable by the patient table 30 and the positioning unit 36 of the patient table 30 into the scanning region. Typically, the field magnet 11 is a superconducting magnet that may provide magnetic fields with a magnetic flux density of up to 3T and in the newest devices even higher. For weaker field strengths, however, permanent magnets or electromagnets with normally conducting coils may also be used.

The magnet unit 10 further includes gradient coils 12 that are configured, for spatial differentiation of the acquired mapping regions in the examination volume, to overlay variable magnetic fields onto the magnetic field B0 in three spatial directions. The gradient coils 12 may be coils made of normally conducting wires that may generate mutually orthogonal fields in the examination volume.

The magnet unit 10 also includes a body coil 14 that is configured to emit a radio frequency signal fed via a signal line 33 into the examination volume and to receive resonance signals emitted from the patient 100 and to pass them on via a signal line. However, the body coil 14 may be replaced, for the emission of the radio frequency signals and/or the reception, by local coils 50 that are arranged in the patient tunnel 16 close to the patient 100. The local coil 50 may also be configured for transmitting and receiving and therefore a body coil 14 may be omitted.

A control unit 20 supplies the magnet unit 10 with the different signals for the gradient coils 12 and the body coil 14 and evaluates the received signals. A magnetic resonance tomograph control system 23 thereby coordinates the subsidiary units.

Thus, the control unit 20 includes a gradient controller 21 that is configured to supply the gradient coils 12 via feed lines with variable currents that provide the desired gradient fields in the examination volume in a temporally coordinated manner.

Furthermore, the control unit 20 includes a radio frequency unit 22 that is configured to generate a radio frequency pulse with a pre-determined temporal sequence, amplitude, and spectral power distribution for the excitation of a magnetic resonance of the nuclear spins in the patient 100. Therein, pulse power levels in the kilowatt range may be achieved. The individual units are connected to one another via a signal bus 25.

The radio frequency signal generated by the radio frequency unit 22 is fed via a signal connection of the body coil 14 and is emitted into the body of the patient 100 in order to excite the nuclear spins there. An emission of the radio frequency signal via one or more local coils 50 may be used.

The local coil 50 then receives a magnetic resonance signal from the body of the patient 100 since, due to the small distance, the signal-to-noise ratio (SNR) of the local coil 50 is better than with a reception by the body coil 14. The MR signal received by the local coil 50 is processed in the local coil 50 and passed on to the radio frequency unit 22 of the magnetic resonance tomography system 1 for evaluation and image acquisition. The transfer takes place in the local coil in a wireless or line-bound manner. Similarly, control signals, for example a detuning signal must be transferred to the local coil 50 during the excitation pulse to detune the local coil 50. This may take place in the case of a line-bound local coil 50 via a control line, in the case of a wireless local coil 50, for example, by way of a magnetic alternating field that the magnetic resonance tomography system 1 generates and is received by an induction coil in the local coil 50. A transfer by way of an electromagnetic wave may be used, for example a transfer in an ISM band or with commercial radio technology such as Bluetooth or WLAN.

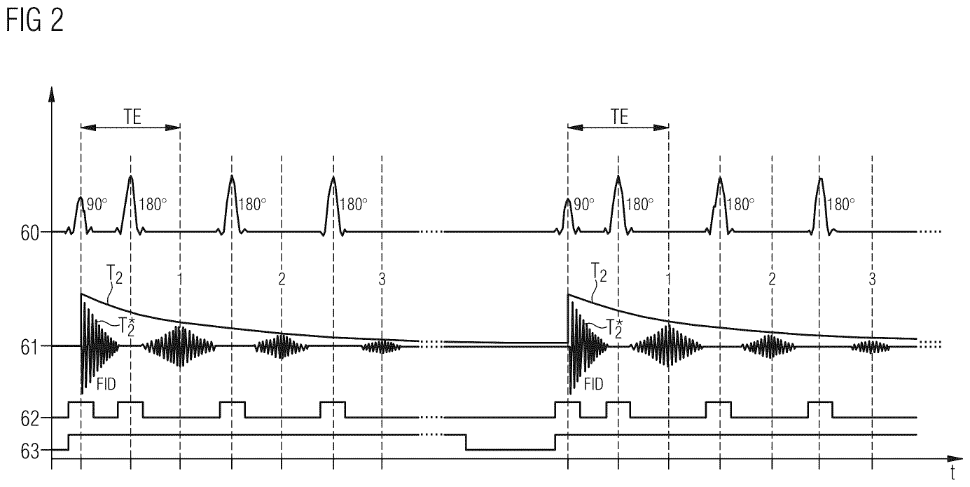

FIG. 2 depicts a schematic representation of a temporal progression of an exemplary image acquisition sequence. The abscissa indicates the time in arbitrary time units. Along the ordinate, the essential signals of an image acquisition sequence are plotted. From top to bottom, these are:

The excitation pulses 60, wherein the numbers in the figure give the deflection of the spin in degrees that is to be achieved by the excitation pulses 60. 90 degrees corresponds herein to the maximum deflection as the excitation pulse and 180 degrees is a spin flip to generate a spin echo.

The magnetic resonance signal 61 to be acquired by the local coil has a maximum amplitude after the 90 degree excitation pulse and then decays with a time constant T1 through dephasing until it is rephased again by the spin flip. The amplitude of the spin echo decays exponentially with the time constant T2, so that after a multiple of T2, no usable magnetic resonance signal is to be acquired.

The detuning signal 62, wherein here the detuning signal 62 is the signal transferred by the magnetic resonance tomography system 1 to the local coil 50 and indicates when the local coil 1 should be detuned so that the excitation pulses 60 cause no damage to the local coil 50 or endanger the patient 100.

The energy control signal 63 specifies the signal that an energy usage control system 56 of the local coil 50 outputs to functional units of the local coil 50 in order to set it into an energy-saving operational state.

In the embodiment shown in FIG. 2, the energy control signal 63 specifies an operational state with a raised energy usage for acquiring the magnetic resonance signals for a duration determined substantially by T2 before the functional units are set into an energy-saving operational state. The energy-saving operational state is ended with the next detuning signal 62, that accompanies the next excitation pulse 60.

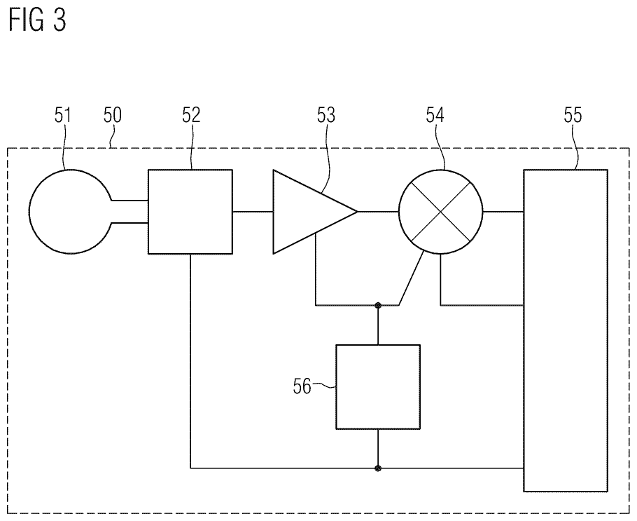

In FIG. 3, by way of example, the essential units involved in a wireless local coil 50 that are required to implement a temporal progression according to FIG. 2 are shown. Energy stores such as a battery or details of a wireless transfer are not shown in detail here.

In a line-bound embodiment of the local coil, analogously, the wireless transfer unit 55 may be replaced by a line-bound transfer, in the simplest case by a cable and plug connection for the individual signals that are to be transferred.

The local coil 50 includes an antenna coil 51 for receiving magnetic resonance signals. The antenna coil 51 may be brought from a detuning apparatus 52 coupled thereto into a non-resonant state, so that excitation pulses of the magnetic resonance tomography system 1 do not cause damage. The detuning apparatus 52 contains the detuning signal from the wireless transfer unit 55 that provides for a wireless signal transfer from and to the magnetic resonance tomography system 1.

Received magnetic resonance signals are amplified by an amplifier/LNA 53, converted by a mixer 54 to an intermediate frequency and transferred from the wireless transfer unit 55 to the magnetic resonance tomography system 1.

A mixing frequency for the frequency conversion may be provided by the magnetic resonance tomography system 1 via the transfer unit 55.

The transfer unit 55 may also have different functional groups for the different signals. For example, a transfer of the magnetic resonance signals may take place similarly at the intermediate frequency. The detuning signal may be induced by a magnetic alternating field in a receiving coil of the transfer unit 55, and the same is conceivable for the mixed signal. Also conceivable, however, is a digital transfer of all the signals.

An energy usage control system 56 also receives the detuning signal and, dependent upon the detuning signal, passes an energy usage signal on to the functional units of mixer 54 and LNA 53. In order to provide the energy usage signal as shown in FIG. 2, not only for the duration of the detuning signal, but for a predetermined time in the order or magnitude of T2, the energy usage control system 56 has a time member, for example, a monoflop or a timer.

The energy control signal is implemented by the functional units such that in the absence of an energy control signal, an energy-saving operational state is assumed by the functional units. There are corresponding components such as, for example, amplifiers from Texas Instruments that have a power-down mode. The energy usage control system 56 may directly interrupt an energy supply of one or more functional units.

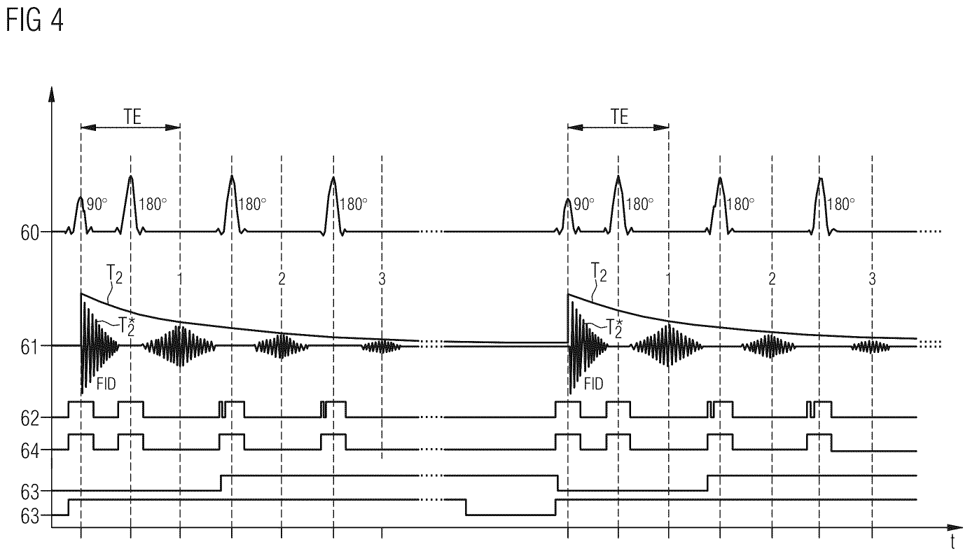

FIG. 4 depicts a schematic representation of a temporal progression of an exemplary image acquisition sequence. The temporal progression in FIG. 2 and FIG. 4 differs in that a plurality of different operational states are set at different time points, as illustrated by two energy control signals 63. The energy supply control system 56 may include a common line or bus via which the energy usage control system 56 may communicate different operational states to the functional units.

In order to communicate different states from the magnetic resonance tomography system 1 to the local coil 50 independently or in addition to the detuning signal, as shown in FIG. 4, the detuning signal may be encoded with a message, for example, by way of short interruptions. As set out below, the energy usage control system 56 produces a detuning output signal 64 without interruptions for the detuning apparatus 52.

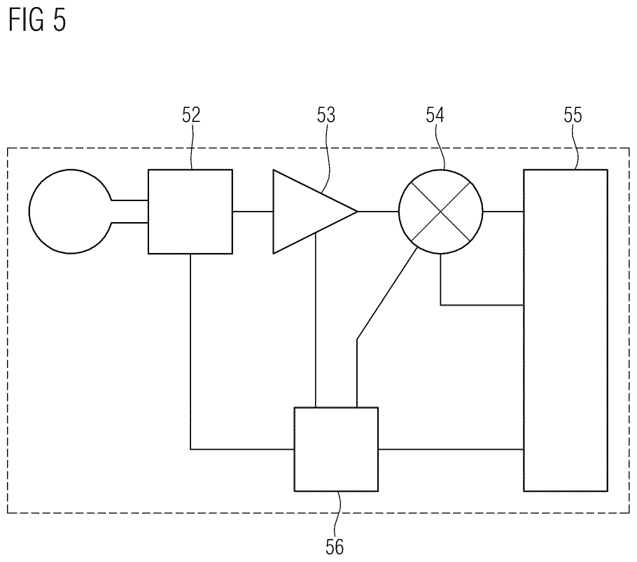

FIG. 5 depicts a schematic detailed representation of an embodiment of a local coil 50, that uses and implements the temporal progression of FIG. 4.

The embodiment of FIG. 5 differs firstly from the embodiment of FIG. 3 in that the energy usage control system 56 does not merely receive the encoded detuning signal fed to it from the magnetic resonance tomography system 1 that is then also passed on in the same form to the detuning apparatus 52. Rather, the energy usage control system 56 has a decoder that recognizes the encoding and acquires the encoded message. Depending upon the message, the energy usage control system 56 transmits energy control signals 63 to one or more functional units via one or more signal connections.

Furthermore, the decoder again creates a detuning output signal 64 without the short interruptions and passes it on to the detuning apparatus 52. It is thereby ensured that the antenna coil 51 is detuned without interruption.

In the example of FIG. 4 and FIG. 5, the LNA is used in two different operating modes for reception. In a starting phase after the 90 degree excitation pulse, the magnetic resonance signals have a large amplitude that, for linear processing, requires a high bias current from the LNA. If the magnetic resonance signals fall below a threshold value due to the decay of the excitation, the optimum processing may be ensured with a small bias current and thus also a small energy usage. The magnetic resonance tomography system 1 and/or its control system 23 signals this by way of a short interruption in the detuning signal 62 that may be recognized, for example, by way of a high-pass filter, a logic circuit, or a processor. The energy usage control system 56 generates a separate control signal for the LNA 53 therefrom in order to set this operational state with a reduced bias. This may be created, for example, by way of a monoflop or a bistable flipflop, that is then reset again by way of the detuning signal in AND-connection to the inverted second energy control signal. This implementation by way of simple logic circuits itself advantageously has a low energy usage, generates hardly any interfering emissions, and may be integrated into an existing environment, for example also with a wireless local coil 50 having analog transfer, without any great alterations.

More complex variants may be used, for example with digital transfer, in which the magnetic resonance tomography system 1 also transfers the operational states separately to the local coil 50 without altering the detuning signal.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A local coil for a magnetic resonance tomography system, the local coil comprising:
a detuning apparatus with a signal path for activation;
a functional unit having two operational states with differing energy usage; and
an energy usage control system that is configured to control the operational states of the functional unit dependent upon an image acquisition.

2. The local coil of claim 1, wherein the energy usage control system is configured to acquire a detuning signal for the detuning apparatus on the signal path and to control the operational states of the functional unit dependent upon the detuning signal.

3. The local coil of claim 2, wherein the energy usage control system includes a decoder which is configured to decode a plurality of messages encoded in the detuning signal and to set a plurality of operational states of the functional unit dependent upon the messages.

4. The local coil of claim 1, wherein the energy usage control system is configured, in an absence of a predetermined event of the image acquisition, to set the functional unit in a predetermined operational state.

5. The local coil of claim 1, further comprising:
a magnetic field sensor in signal connection with the energy usage control system, wherein the magnetic field sensor is configured to acquire a gradient field, wherein the energy usage control system is configured to set the operational states dependent upon a signal from the magnetic field sensor.

6. A magnetic resonance tomography system comprising:
a local coil comprising:
a detuning apparatus with a signal path for activation;
a functional unit having two operational states with differing energy usage; and
an energy usage control system that is configured to control the operational states of the functional unit dependent upon an image acquisition, wherein the energy usage control system is configured to acquire a detuning signal for the detuning apparatus on the signal path and to control the operational states of the functional unit dependent upon the detuning signal, wherein the energy usage control system includes a decoder which is configured to decode a plurality of messages encoded in the detuning signal and to set a plurality of operational states of the functional unit dependent upon the messages, wherein the energy usage control system is configured, in an absence of a predetermined event of the image acquisition, to set the functional unit in a predetermined operational state; and
an encoding unit configured to encode a message regarding an operational state of the functional unit in the detuning signal.
